(19) United States
(12) Patent Application Publication
(10) Pub. No.: US 2026/0215660 A1
(43) Pub. Date: Jul. 30, 2026

(54) MEDICAL SUPPORT DEVICE, ENDOSCOPE SYSTEM, MEDICAL SUPPORT METHOD, AND PROGRAM

(71) Applicant: FUJIFILM Corporation, Tokyo (JP)

(72) Inventor: Rito MURASE, Tokyo (JP)

(73) Assignee: FUJIFILM Corporation, Tokyo (JP)

(21) Appl. No.: 19/441,757

(22) Filed: Jan. 6, 2026

(30) Foreign Application Priority Data
Jan. 28, 2025 (JP) 2025-012404

(51) Int. Cl. A61B 1/00; A61B 1/31

(52) U.S. Cl. CPC A61B 1/000094; A61B 1/00006; A61B 1/000096; A61B 1/00043; A61B 1/31

(57) ABSTRACT

A medical support device includes a processor. The processor generates, based on an image obtained by imaging an interior portion of a luminal organ, positional information that enables identification of a lumen position that is a position in the image of a lumen shown in the image. The processor outputs visible information that enables visual identification of the lumen position in the image on a screen based on the positional information. The processor suppresses output of the visible information based on an amount of deviation about a center of the image between the lumen position identified from reference positional information that is the positional information and the lumen position identified from subsequent positional information that is the positional information generated after the reference positional information.

### CROSS-REFERENCE TO RELATED APPLICATIONS

This application claims priority under 35 USC 119 from Japanese Patent Application No. 2025-012404 filed on January 28, 2025, the disclosure of which is incorporated by reference herein.

### BACKGROUND

### 1. Technical Field

The present disclosure relates to a medical support device, an endoscope system, a medical support method, and a program.

### 2. Related Art

WO2024/095675A discloses a medical support device comprising a processor. In the medical support device disclosed in WO2024/095675A, the processor acquires intestinal direction-related information related to an intestinal direction of a duodenum in which an endoscope is inserted, based on geometric characteristic information that enables identification of geometric characteristics of the duodenum, and outputs the intestinal direction-related information.

In addition, in the medical support device disclosed in WO2024/095675A, the geometric characteristic information includes an intestinal wall image obtained by imaging an intestinal wall of the duodenum with a camera provided in the endoscope, and the processor acquires the intestinal direction-related information by executing first image recognition processing on the intestinal wall image.

In addition, in the medical support device disclosed in WO2024/095675A, the intestinal direction-related information includes first direction information that enables identification of a first direction intersecting the intestinal direction at a predetermined angle. The first direction information is obtained by executing second image recognition processing on the intestinal wall image obtained by imaging the intestinal wall of the duodenum with the camera provided in the endoscope. The first direction information is information obtained with a confidence level of equal to or higher than a threshold value by performing AI-based image recognition processing as the second image recognition processing.

WO2024/018713A discloses an image processing device comprising a processor. In the image processing device disclosed in WO2024/018713A, the processor from an image in accordance with a trained model obtained through machine learning based on a positional relationship between a plurality of divided regions obtained by dividing an image obtained by imaging a tubular organ with a camera provided in the endoscope and a lumen correspondence region included in the image, and acquires a luminal direction that is a direction in which an endoscope is inserted, outputs luminal direction information that is information indicating the luminal direction.

### SUMMARY

One embodiment according to the present disclosure provides a medical support device, an endoscope system, a medical support method, and a program that can prevent an observer who observes visible information on a screen from being confused due to a large change in position of a lumen visually identified from the visible information.

A first aspect according to the present disclosure relates to a medical support device comprising: a processor configured to: generate, based on an image obtained by imaging an interior portion of a luminal organ, positional information that enables identification of a lumen position that is a position in the image of a lumen shown in the image; output visible information that enables visual identification of the lumen position in the image on a screen based on the positional information; and suppress output of the visible information based on an amount of deviation about a center of the image between the lumen position identified from reference positional information that is the positional information and the lumen position identified from subsequent positional information that is the positional information generated after the reference positional information.

A second aspect according to the present disclosure relates to the medical support device according to the first aspect, in which the positional information is generated by a trained model by inputting the image to the trained model.

A third aspect according to the present disclosure relates to the medical support device according to the first aspect or the second aspect, in which the output of the visible information is suppressed in a case where the amount of deviation exceeds a threshold value.

A fourth aspect according to the present disclosure relates to the medical support device according to the third aspect, in which the output of the visible information is suppressed from when the amount of deviation exceeds the threshold value until a predetermined condition is satisfied.

A fifth aspect according to the present disclosure relates to the medical support device according to the fourth aspect, in which the predetermined condition includes a condition in which the lumen position identified from each piece of the positional information generated based on each of a plurality of the images adjacent in time falls within a predetermined range about the center for a designated period.

A sixth aspect according to the present disclosure relates to the medical support device according to the fifth aspect, in which the period is a period during which visual continuity of the visible information is maintained in a case where an output state in which the visible information is output transitions to an output suppression state in which the output of the visible information is suppressed, and then is returned to the output state.

A seventh aspect according to the present disclosure relates to the medical support device according to the fifth aspect, in which the period is determined based on the amount of deviation.

An eighth aspect according to the present disclosure relates to the medical support device according to any one of the fourth to seventh aspects, in which the processor is configured to output the visible information based on the positional information generated in a case where the predetermined condition is satisfied, in a case where the lumen position identified from the positional information generated after the predetermined condition is satisfied deviates from the lumen position identified from the positional information generated in a case where the predetermined condition is satisfied by an amount exceeding the threshold value about the center.

A ninth aspect according to the present disclosure relates to the medical support device according to any one of the third to eighth aspects, in which the threshold value is 90 degrees.

A tenth aspect according to the present disclosure relates to the medical support device according to any one of the third to ninth aspects, in which the case where the amount of deviation exceeds the threshold value refers to a case where a first vector that enables identification of a direction from the center toward the lumen position identified from the reference positional information and a second vector that enables identification of a direction from the center toward the lumen position identified from the subsequent positional information have vector components that are opposite in direction.

An eleventh aspect according to the present disclosure relates to the medical support device according to any one of the third to tenth aspects, in which the case where the amount of deviation exceeds the threshold value refers to a case where a first partitioned region that is a partitioned region to which the lumen position identified from the reference positional information belongs among a plurality of partitioned regions obtained by partitioning the image along the center and a second partitioned region that is a partitioned region to which the lumen position identified from the subsequent positional information belongs among the plurality of partitioned regions deviate from each other by an amount exceeding 90 degrees.

A twelfth aspect according to the present disclosure relates to the medical support device according to the eleventh aspect, in which each of the plurality of partitioned regions is a region obtained by radially partitioning the image.

A thirteenth aspect according to the present disclosure relates to the medical support device according to the twelfth aspect, in which the plurality of partitioned regions are eight radially partitioned regions.

A fourteenth aspect according to the present disclosure relates to the medical support device according to any one of the first to thirteenth aspects, in which the processor is configured to output reliability identification information that enables visual identification of reliability of the visible information based on the amount of deviation.

A fifteenth aspect according to the present disclosure relates to the medical support device according to the fourteenth aspect, in which the processor is configured to output information that enables visual identification of a fact that the reliability is lower than a certain level as the reliability identification information in a case where the amount of deviation exceeds the threshold value.

A sixteenth aspect according to the present disclosure relates to the medical support device according to any one of the first to fifteenth aspects, in which the output of the visible information is achieved by displaying the visible information on the screen.

A seventeenth aspect according to the present disclosure relates to the medical support device according to any one of the first to sixteenth aspects, in which the image is an endoscopic image generated by imaging the interior portion of the luminal organ including the lumen with an endoscope.

An eighteenth aspect according to the present disclosure relates to an endoscope system comprising: the medical support device according to any one of the first to seventeenth aspects; and an endoscope, in which the image is generated by imaging the interior portion of the luminal organ including the lumen with the endoscope.

A nineteenth aspect according to the present disclosure relates to a medical support method comprising: generating, based on an image obtained by imaging an interior portion of a luminal organ, positional information that enables identification of a lumen position that is a position in the image of a lumen shown in the image; outputting visible information that enables visual identification of the lumen position in the image on a screen based on the positional information; and suppressing output of the visible information based on an amount of deviation about a center of the image between the lumen position identified from reference positional information that is the positional information and the lumen position identified from subsequent positional information that is the positional information generated after the reference positional information.

A twentieth aspect according to the present disclosure relates to a program causing a computer to execute comprising: generating, based on an image obtained by imaging an interior portion of a luminal organ, positional information that enables identification of a lumen position that is a position in the image of a lumen shown in the image; outputting visible information that enables visual identification of the lumen position in the image on a screen based on the positional information; and suppressing output of the visible information based on an amount of deviation about a center of the image between the lumen position identified from reference positional information that is the positional information and the lumen position identified from subsequent positional information that is the positional information generated after the reference positional information.

### BRIEF DESCRIPTION OF THE DRAWINGS

Exemplary embodiments of the technology of the disclosure will be described in detail based on the following figures, wherein:

FIG. 1 is a conceptual diagram illustrating an example of an aspect in which an endoscope system is used by a doctor;

FIG. 2 is a conceptual diagram illustrating an example of an overall configuration of the endoscope system;

FIG. 3 is a block diagram illustrating an example of a hardware configuration of an electrical system of the endoscope system;

FIG. 4 is a block diagram illustrating an example of main functions of a processor included in a medical support device and an example of information stored in a storage;

FIG. 5 is a block diagram illustrating an example of a hardware configuration of an electrical system of an information processing device;

FIG. 6 is a conceptual diagram illustrating an example of an aspect in which training data is generated by the information processing device;

FIG. 7 is a conceptual diagram illustrating an example of an example image;

FIG. 8 is a conceptual diagram illustrating an example of training data generated in a case where a lumen is shown in one of a plurality of partitioned regions obtained by radially partitioning the example image illustrated in FIG. 7;

FIG. 9 is a conceptual diagram illustrating an example of processing contents in the information processing device in a case where the lumen recognition model is generated by training a model through machine learning using the training data;

FIG. 10 is a conceptual diagram illustrating an example of processing contents of lumen recognition processing performed by a recognition unit and an example of processing contents of a controller;

FIG. 11 is a conceptual diagram illustrating an example of an aspect in which a mark is superimposed and displayed on a frame in a case where an amount of deviation between lumen positions identified from positional information adjacent in time generated by the recognition unit is equal to or less than a threshold value, and the mark is hidden in a case where the amount of deviation exceeds the threshold value;

FIG. 12 is a flowchart illustrating an example of a flow of medical support processing;

FIG. 13 is a conceptual diagram illustrating a form example in which the mark is not displayed on the screen in a case where a state where the lumen position falls within a predetermined range continues for a designated period;

FIG. 14 is a conceptual diagram illustrating an example of an aspect in which the mark generated based on the positional information generated last in the designated period illustrated in FIG. 13 is superimposed and displayed on the frame in a case where the amount of deviation between the lumen position identified from the positional information generated last in the designated period illustrated in FIG. 13 and the lumen position identified from the positional information generated first after the designated period illustrated in FIG. 13 exceeds the threshold value;

FIG. 15 is a conceptual diagram illustrating an example of an aspect in which it is determined whether or not the amount of deviation exceeds the threshold value using a vector instead of the partitioned region, and the mark is displayed or hidden based on a determination result; and

FIG. 16 is a conceptual diagram illustrating an example of a series of processing in which a processor included in a computer gives a processing execution request to an external device via a network, the external device executes processing in response to the processing execution request, and the processor included in the computer receives a processing result from the external device.

### DETAILED DESCRIPTION

Hereinafter, examples of embodiments of a medical support device, an endoscope system, a medical support method, and a program according to the present disclosure will be described with reference to the accompanying drawings. In addition, the present disclosure is also applicable to a program and a computer program product.

First, the terms used in the following description will be described.

CPU is an abbreviation for "central processing unit". GPU is an abbreviation for "graphics processing unit". GPGPU is an abbreviation for "general-purpose computing on graphics processing units". NPU is an abbreviation for "neural processing unit". APU is an abbreviation for "accelerated processing unit". TPU is an abbreviation for "tensor processing unit". RAM is an abbreviation for "random-access memory". ASIC is an abbreviation for "application-specific integrated circuit". PLD is an abbreviation for "programmable logic device". SPLD is an abbreviation for "simple programmable logic device". CPLD is an abbreviation for "complex programmable logic device". FPGA is an abbreviation for "field-programmable gate array". SoC is an abbreviation for "system-on-a-chip". SSD is an abbreviation for "solid-state drive". HDD is an abbreviation for "hard disk drive". CD-ROM is an abbreviation for "compact disc read only memory". DVD-ROM is an abbreviation for "digital versatile disc read only memory". USB is an abbreviation for "Universal Serial Bus". EL is an abbreviation for "electro-luminescence". CMOS is an abbreviation for "complementary metal oxide semiconductor". CCD is an abbreviation for "charge coupled device". FIFO is an abbreviation for "first in first out". AI is an abbreviation for "artificial intelligence". WLI is an abbreviation for "white light imaging". BLI is an abbreviation for "blue light imaging". LCI is an abbreviation for "linked color imaging". NBI is an abbreviation for "narrow band imaging". I/F is an abbreviation for "interface". LAN is an abbreviation for "local area network". WAN is an abbreviation for "wide area network". 5G is an abbreviation for "5th generation mobile communication system".

Hereinafter, a processor with a reference numeral (hereinafter, simply referred to as a "processor") may be one computing device or may be a combination of a plurality of computing devices. In addition, the processor may be one type of computing device or may be a combination of a plurality of types of computing devices. Examples of the computing device include a CPU, a GPU, a GPGPU, an NPU, an APU, and a TPU.

In the following description, a memory with a reference numeral is a memory, such as a RAM, that temporarily stores information, and is used by the processor as a work memory.

Hereinafter, a storage with a reference numeral is one or a plurality of non-volatile storage devices that store various programs, various parameters, and the like. Examples of the non-volatile storage device include a data storage device (for example, an SSD) made using a flash memory, an HDD, and a magnetic tape device. Furthermore, examples of the storage include a cloud storage.

In the following embodiment, an external I/F with a reference numeral controls the transmission and reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), and Bluetooth (registered trademark).

In the following embodiment, the expression "A and/or B" is synonymous with the expression "at least one of A or B". That is, the phrase "A and/or B" may mean only A, may mean only B, or may mean a combination of A and B. Additionally, in the present specification, the same concept as the expression "A and/or B" is applied to a case where the connection of three or more matters is expressed by "and/or".

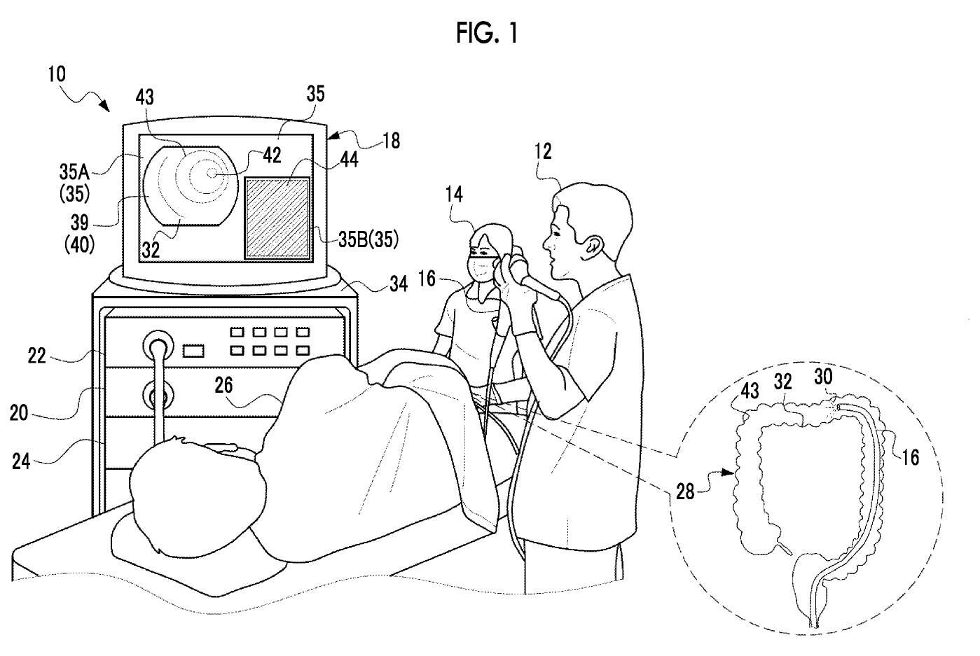

FIG. 1 is a conceptual diagram illustrating an example of an aspect in which an endoscope system 10 is used. As illustrated in FIG. 1, an endoscope system 10 is used by a doctor 12 in an endoscopy and the like. A staff member 14 such as a nurse assists with the endoscopy.

The endoscope system 10 is communicably connected to a communication device (not illustrated), and information obtained by the endoscope system 10 is transmitted to the communication device. Examples of the communication device include a server, a personal computer, and/or a tablet terminal that manage various types of information such as electronic medical records. The communication device receives the information transmitted from the endoscope system 10, and executes processing using the received information (for example, processing of storing the information in the electronic medical record).

The endoscope system 10 comprises an endoscope 16, a display device 18, a light source device 20, a control device 22, and a medical support device 24. In the present embodiment, the endoscope system 10 is an example of an "endoscope system" according to the present disclosure, the endoscope 16 is an example of an "endoscope" according to the present disclosure, and the medical support device 24 is an example of a "medical support device" according to the present disclosure.

The endoscope system 10 is a modality for performing a medical examination on a large intestine 28, which is a luminal organ included in a body of a subject 26 (for example, a patient), using the endoscope 16. The large intestine 28 in the present embodiment is a target to be observed by the doctor 12.

The endoscope 16 is used by the doctor 12 and is inserted into the body of the subject 26. In the present embodiment, the endoscope 16 is inserted into the large intestine 28 of the subject 26. The large intestine 28 in the present embodiment is an example of a "luminal organ" according to the present disclosure.

The endoscope system 10 images an interior portion of the large intestine 28 including a lumen 42 by using the endoscope 16 inserted into the large intestine 28 of the subject 26, and performs various medical treatments on the large intestine 28 as necessary.

The large intestine 28 has the lumen 42. The endoscope 16 is inserted into the lumen 42. A position of the lumen 42 in the large intestine 28 can be medically identified based on a form pattern of a plurality of folds 43 (for example, the shape, the orientation, and the like of the plurality of folds 43) which are characteristic regions in the large intestine 28. In the present embodiment, although the details will be described later, the position of the lumen 42 is recognized by AI that has been trained using various types of information such as the form pattern of the plurality of folds 43 through machine learning, and a recognition result is provided as visually ascertainable information to the doctor 12. The lumen 42 in the present embodiment is an example of a "lumen" according to the present disclosure.

The endoscope system 10 acquires an image showing an aspect including the lumen 42 in the large intestine 28 by imaging the interior portion of the large intestine 28 including the lumen 42, and outputs the acquired image. In the present embodiment, the endoscope system 10 has an optical imaging function of emitting light 30 in the large intestine 28 and imaging reflected light obtained by being reflected by an intestinal wall 32 of the large intestine 28.

Here, the endoscopy of the large intestine 28 has been described as an example, but this is merely an example, and the present disclosure is applicable to the endoscopy of a luminal organ such as the esophagus, the stomach, the duodenum, or the trachea.

The light source device 20, the control device 22, and the medical support device 24 are installed in a wagon 34. The wagon 34 is provided with a plurality of tables in an up-down direction, and the medical support device 24, the light source device 20, and the control device 22 are installed from a lower table to an upper table. Moreover, the display device 18 is installed on the uppermost table in the wagon 34.

The control device 22 controls the entire endoscope system 10. The control device 22 executes various types of processing on the image obtained by imaging the intestinal wall 32 with the endoscope 16. In addition, the medical support device 24 executes AI-based processing and the like on the image that has been subjected to various types of processing by the control device 22, under the control of the control device 22, and outputs various types of information including a processing result of the AI-based processing or the like. Examples of the output destination of the various types of information include the display device 18, a stationary storage medium (for example, a storage mounted in the endoscope system 10, a storage of a server or the like that is connected to the endoscope system 10 in a communicable manner, and the like), and/or a portable storage medium (for example, a memory card, a USB flash drive, and the like).

The display device 18 displays various types of information (as an example, various types of information output from the medical support device 24). Examples of the display device 18 include a liquid crystal display and an EL display. A tablet terminal equipped with a display may be used instead of or together with the display device 18.

The display device 18 displays a screen 35. A plurality of display regions are included in the screen 35. The plurality of display regions are arranged in the screen 35. In the example illustrated in FIG. 1, a first display region 35A and a second display region 35B are illustrated as examples of the plurality of display regions. A size of the first display region 35A is larger than a size of the second display region 35B. The first display region 35A is used as the main display region, and the second display region 35B is used as the sub-display region. The size relationship between the first display region 35A and the second display region 35B is not limited to this, and need only be a size relationship that can be included within the screen 35.

An endoscopic video image 39 is displayed in the first display region 35A. The endoscopic video image 39 is obtained by executing various types of processing on a plurality of images arranged in time series obtained by imaging the interior portion of the large intestine 28 of the subject 26 with the endoscope 16. The intestinal wall 32 shown in the endoscopic video image 39 includes the lumen 42 as the region of interest (that is, an observation target region) at which the doctor 12 gazes, and the doctor 12 can visually recognize the aspect of the intestinal wall 32 including the lumen 42 through the endoscopic video image 39.

The image displayed in the first display region 35A is one frame 40 included in a video image including a plurality of frames 40 arranged in time series. That is, the plurality of frames 40 arranged in time series are displayed in the first display region 35A at predetermined frame rates (for example, a dozen frames per second or a few dozen frames per second). The frame 40 in the present embodiment is an example of an "image" and an "endoscopic image" according to the present disclosure.

Examples of the video image displayed in the first display region 35A include a video image in a live view mode. The live view mode is merely an example, and the video image may be a video image temporarily stored in a memory or the like and then displayed, such as a video image in a post view mode. Further, each frame included in a video image for recording stored in the memory or the like may be reproduced and displayed as the endoscopic video image 39 on the screen 35 (for example, in the first display region 35A).

The second display region 35B is displayed at the lower right of the screen 35 in the front view. The second display region 35B may be displayed at any position as long as the position is within the screen 35 of the display device 18, but is preferably displayed at a position that can be compared with the endoscopic video image 39. Auxiliary information 44 for assistance of the doctor 12 in a medical determination or the like is displayed in the second display region 35B. The auxiliary information 44 is information to be referred to by the doctor 12. Examples of the auxiliary information 44 include various types of information on the subject 26 into whom the endoscope 16 is inserted and/or various types of information obtained by executing medical support processing described later. The screen 35 in the present embodiment is an example of a "screen" according to the present disclosure.

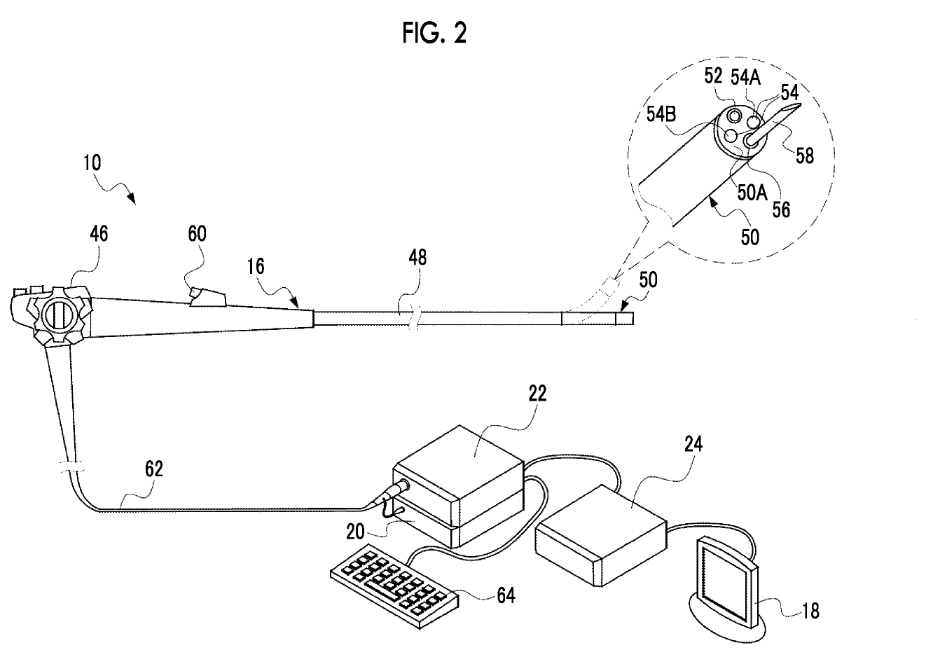

FIG. 2 is a conceptual diagram illustrating an example of an overall configuration of the endoscope system 10. As illustrated in FIG. 2, the endoscope 16 comprises an operating part 46 and an insertion part 48. The insertion part 48 is formed in a tubular shape, and is partially curved by operating the operating part 46. The insertion part 48 is inserted into the large intestine 28 while being curved along the shape of the large intestine 28 (see FIG. 1) in accordance with the operation of the operating part 46 performed by the doctor 12 (see FIG. 1).

A camera 52, an illumination device 54, and a treatment tool opening 56 are provided at a distal end portion 50 of the insertion part 48. A portion of the camera 52 (for example, an imaging optical system) and a portion of the illumination device 54 (for example, an irradiation optical system) are exposed from a distal end surface 50A of the distal end portion 50.

The camera 52 is mounted in the endoscope 16 and is inserted into the body cavity (here, as an example, the lumen 42) of the subject 26 to image the observation target region. Examples of the camera 52 include a CMOS camera. However, this is merely an example, and the camera 52 may be other types of cameras such as CCD cameras. In the present embodiment, the camera 52 generates an image showing the aspect including the lumen 42 in the large intestine 28 by imaging the interior portion of the large intestine 28 including the lumen 42. The image generated by the camera 52 is an image of which an outer shape is circular. For example, the image generated by the camera 52 is processed by the control device 22 into a shape in which an upper end portion and a lower end portion are masked. Accordingly, as illustrated in FIG. 1, an image in which an upper end edge and a lower end edge are linear and a left side edge and a right side edge are arc-shaped is generated as the frame 40.

The illumination device 54 includes illumination windows 54A and 54B. The illumination windows 54A and 54B are provided on the distal end surface 50A. The illumination device 54 emits the light 30 (see FIG. 1) through the illumination windows 54A and 54B. Examples of the type of the light 30 emitted from the illumination device 54 include WLI light (for example, white light), LCI light (for example, light obtained by combining red light, green light, and blue light), BLI light (for example, blue light), and/or NBI light (for example, light obtained by combining blue light and green light). The camera 52 images the interior portion of the large intestine 28 using an optical method in a state where the interior portion of the large intestine 28 is irradiated with the light 30 (see FIG. 1) by the illumination device 54.

The treatment tool opening 56 is an opening for allowing a treatment tool 58 to protrude from the distal end portion 50. Moreover, the treatment tool opening 56 is also used as a suction port for suctioning blood, internal contaminants, and the like and a sending-out port for sending out fluid. Examples of the fluid include gas (for example, air or the like) and/or liquid (for example, water or the like).

A treatment tool insertion port 60 is formed at the operating part 46, and the treatment tool 58 is inserted into the insertion part 48 through the treatment tool insertion port 60. The treatment tool 58 passes through the insertion part 48 to protrude from the treatment tool opening 56 to the outside. In the example illustrated in FIG. 2, an aspect is illustrated in which a biopsy needle protrudes through the treatment tool opening 56 as the treatment tool 58. Here, although the biopsy needle has been described as an example of the treatment tool 58, this is merely an example, and the treatment tool 58 may be grasping forceps, a papillotomy knife, a snare, a catheter, a guide wire, a cannula, and/or a biopsy needle with a guide sheath.

The endoscope 16 is connected to the light source device 20 and the control device 22 through a universal cord 62. The medical support device 24 and a reception device 64 are connected to the control device 22. Further, the display device 18 is connected to the medical support device 24. That is, the control device 22 is connected to the display device 18 via the medical support device 24.

Here, since the medical support device 24 is used as an example of an external device for expanding the functions of the control device 22, the form example has been described in which the control device 22 and the display device 18 are indirectly connected to each other via the medical support device 24, but this is merely an example. For example, the display device 18 may be directly connected to the control device 22. In this case, for example, the functions of the medical support device 24 need only be installed in the control device 22, or the control device 22 need only have a function of directing a server (not illustrated) to execute the same processing as the processing (for example, the medical support processing which will be described later) executed by the medical support device 24, receiving a processing result obtained by the server, and using the processing result.

The reception device 64 receives the instruction from the doctor 12, and outputs the received instruction as an electric signal to the control device 22. Examples of the reception device 64 include a keyboard, a mouse, a touch panel, a foot switch, a microphone, and/or a remote control device.

The control device 22 controls the light source device 20, transmits and receives various signals to and from the camera 52, or transmits and receives various signals to and from the medical support device 24.

The light source device 20 emits light under the control of the control device 22 and supplies the light 30 (see FIG. 1) to the illumination device 54. A light guide is built in the illumination device 54, and the light 30 supplied from the light source device 20 is emitted from the illumination windows 54A and 54B through the light guide. The control device 22 causes the camera 52 to perform the imaging in a state where the light 30 is emitted from the illumination windows 54A and 54B. The control device 22 generates the plurality of frames 40 arranged in time series by processing the outer shape of the image obtained by imaging performed by the camera 52 or adjusting the image quality or the like of the image. The control device 22 outputs the endoscopic video image 39 including the plurality of generated frames 40 arranged in time series to a predetermined output destination (for example, the medical support device 24).

The medical support device 24 executes various types of processing on the endoscopic video image 39 input from the control device 22 to support a medical treatment (here, for example, endoscopy). The medical support device 24 outputs the endoscopic video image 39 subjected to various types of processing to a predetermined output destination (for example, the display device 18).

Here, although the form example has been described in which the endoscopic video image 39 output from the control device 22 is output to the display device 18 via the medical support device 24, this is merely an example. For example, an aspect may be adopted in which the control device 22 and the display device 18 are connected to each other, and the endoscopic video image 39 subjected to various types of processing by the medical support device 24 is displayed on the display device 18 via the control device 22.

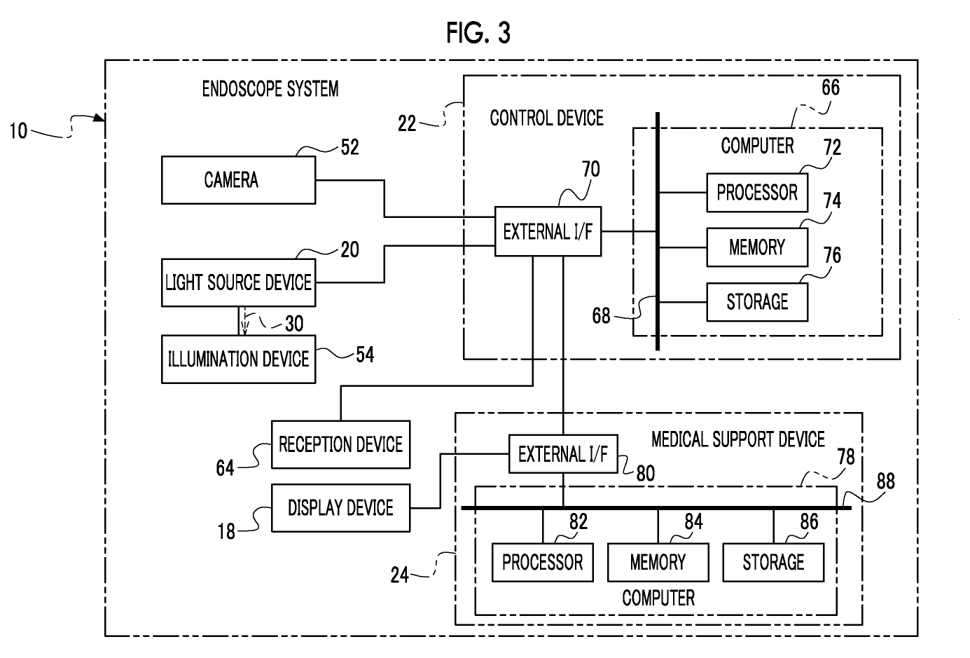

FIG. 3 is a block diagram illustrating an example of a hardware configuration of an electrical system of the endoscope system 10. As illustrated in FIG. 3, the control device 22 comprises a computer 66, a bus 68, and an external I/F 70. The computer 66 comprises a processor 72, a memory 74, and a storage 76. The processor 72, the memory 74, the storage 76, and the external I/F 70 are connected to the bus 68. The processor 72 controls the entire control device 22. The memory 74 and the storage 76 are used by the processor 72.

The external I/F 70 transmits and receives various types of information between one or more devices (hereinafter, also referred to as "first external devices") existing outside the control device 22 and the processor 72.

The camera 52 is connected to the external I/F 70 as one of the first external devices, and the external I/F 70 transmits and receives various types of information between the camera 52 and the processor 72. The processor 72 controls the camera 52 through the external I/F 70. Further, the processor 72 acquires an image generated by imaging the interior portion of the large intestine 28 (see FIG. 1) with the camera 52 via the external I/F 70, and performs various types of processing on the acquired image to generate the endoscopic video image 39 (see FIG. 1).

The light source device 20 is connected to the external I/F 70 as one of the first external devices, and the external I/F 70 transmits and receives various types of information between the light source device 20 and the processor 72. The light source device 20 supplies the light 30 to the illumination device 54 under the control of the processor 72. The illumination device 54 emits the light 30 supplied from the light source device 20.

The reception device 64 is connected to the external I/F 70 as one of the first external devices, and the processor 72 acquires the instruction received by the reception device 64 via the external I/F 70 and executes the processing corresponding to the acquired instruction.

The medical support device 24 comprises a computer 78 and an external I/F 80. The computer 78 comprises a processor 82, a memory 84, and a storage 86. The processor 82, the memory 84, the storage 86, and the external I/F 80 are connected to a bus 88. In the present embodiment, the computer 78 is an example of a "computer" according to the present disclosure, and the processor 82 is an example of a "processor" according to the present disclosure.

The hardware configuration (that is, the processor 82, the memory 84, and the storage 86) of the computer 78 is essentially the same as the hardware configuration of the computer 66, and thus the description of the hardware configuration of the computer 78 will be omitted here.

The external I/F 80 transmits and receives various types of information between one or more devices (hereinafter, also referred to as "second external devices") existing outside the medical support device 24 and the processor 82.

The control device 22 is connected to the external I/F 80 as one of the second external devices. In the example illustrated in FIG. 3, the external I/F 70 of the control device 22 is connected to the external I/F 80. The external I/F 80 transmits and receives various types of information between the processor 82 of the medical support device 24 and the processor 72 of the control device 22. For example, the processor 82 acquires the endoscopic video image 39 (see FIG. 1) from the processor 72 of the control device 22 via the external I/Fs 70 and 80, and executes various types of processing on the acquired endoscopic video image 39. Various types of processing performed by the processor 82 include AI-based processing (for example, lumen recognition processing 166 that is processing using a lumen recognition model 92 described later).

The display device 18 is connected to the external I/F 80 as one of the second external devices. The processor 82 controls the display device 18 via the external I/F 80 such that various types of information (for example, the endoscopic video image 39 that has been subjected to various types of processing) are displayed on the display device 18.

In the endoscopy on the large intestine 28, it may be difficult to smoothly insert the endoscope 16 into the large intestine 28 for various reasons such as a complicated shape of the large intestine 28 or an inexperienced level of technique of the doctor 12. One of specific causes that make it difficult to insert the endoscope 16 into the large intestine 28 is that the doctor 12 loses the position of the lumen 42 to which the distal end (that is, the distal end portion 50 illustrated in FIG. 2) of the endoscope 16 should advance. In order to suppress the occurrence of such a situation, a technique has been developed in which the AI recognizes the position of the lumen 42 shown in the frame 40, and a mark that enables identification of the position of the lumen 42 is superimposed and displayed on the frame 40 displayed on the screen 35.

However, in a case where the AI cannot limit the position of the lumen 42 to one location for a reason such as the AI incorrectly recognizing the position of the lumen 42 shown in the frame 40 (for example, in a case where the AI recognizes two locations present in opposite directions based on the confidence level as the position of the lumen 42 between the frames 40 adjacent in time), the position of the mark displayed on the screen 35 deviates significantly. That is, in a case where the position of the lumen 42 is recognized by the AI for each frame 40, the mark displayed on the screen 35 changes to bounce significantly in a case where the position of the lumen 42 recognized by the AI deviates significantly. In a case where such a phenomenon occurs, it may cause confusion for the doctor 12. It is desirable to address such a problem in order to safely and efficiently advance the endoscopy and various treatments.

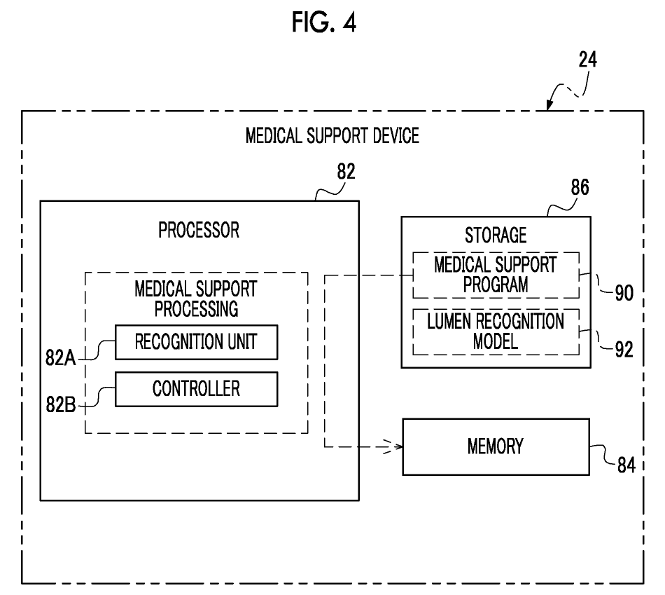

Therefore, in order to address such a problem, in the present embodiment, as illustrated in FIG. 4 as an example, the medical support processing is executed by the processor 82.

FIG. 4 is a block diagram illustrating an example of main functions of the processor 82 included in the medical support device 24 and an example of the information stored in the storage 86. As illustrated in FIG. 4, a medical support program 90 is stored in the storage 86. The medical support program 90 in the present embodiment is an example of a "program" according to the present disclosure.

The processor 82 reads out the medical support program 90 from the storage 86, and executes the readout medical support program 90 on the memory 84 to perform medical support processing. The medical support processing is executed by the processor 82 operating as a recognition unit 82A and a controller 82B in accordance with the medical support program 90 executed on the memory 84.

The lumen recognition model 92 is stored in the storage 86. Although the details will be described later, the lumen recognition model 92 is a machine learning model, which is used by the recognition unit 82A. Examples of the machine learning model include a neural network. Examples of the type of the neural network include a convolutional neural network (for example, ResNet, EfficientNet, U-Net, or the like), a recurrent neural network, and a hybrid model in which the convolutional neural network and the recurrent neural network are combined.

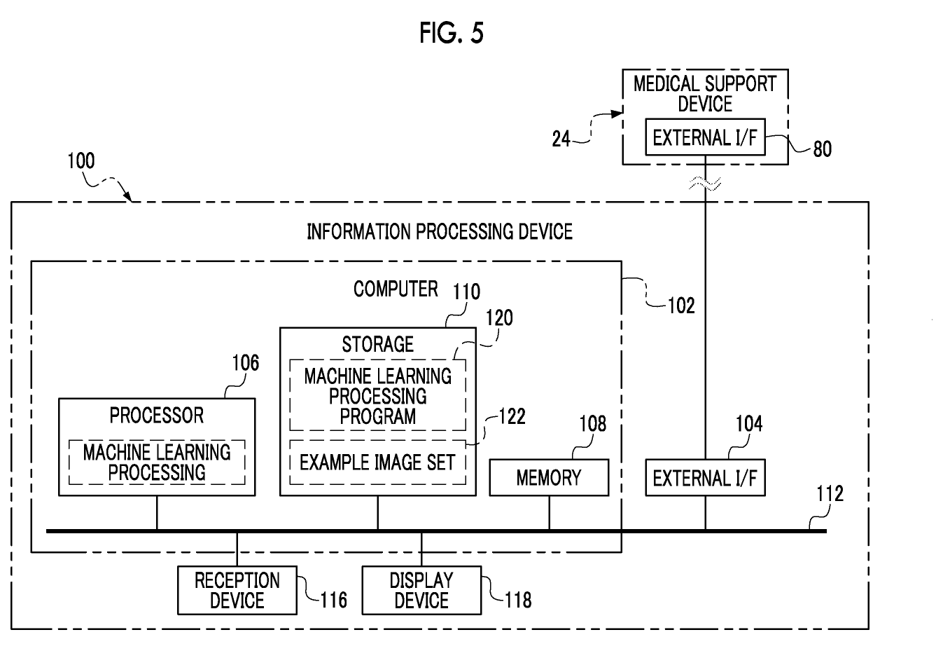

FIG. 5 is a block diagram illustrating an example of a hardware configuration of an electrical system of an information processing device 100 used for generating the lumen recognition model 92. As illustrated in FIG. 5, the information processing device 100 comprises a computer 102 and an external I/F 104. The computer 102 comprises a processor 106, a memory 108, and a storage 110. The processor 106, the memory 108, the storage 110, and the external I/F 104 are connected to a bus 112.

The hardware configuration (that is, the processor 106, the memory 108, and the storage 110) of the computer 102 is essentially the same as the hardware configuration of the computer 66, and thus the description of the hardware configuration of the computer 102 will be omitted here.

The information processing device 100 comprises a reception device 116. The reception device 116 is, for example, a keyboard and/or a mouse, and receives an instruction from a user of the information processing device 100 and the like. The reception device 116 is connected to the bus 112. The processor 106 acquires the instruction received by the reception device 116 and operates in accordance with the acquired instruction.

A display device 118 displays various types of information including the image. Examples of the display device 118 include a liquid crystal display and an EL display. The display device 118 is connected to the bus 112. The processor 106 displays the results obtained by executing various types of processing on the display device 118.

The external I/F 104 transmits and receives various types of information between one or more devices (hereinafter, also referred to as "third external devices") existing outside the information processing device 100 and the processor 106. The medical support device 24 is connected to the external I/F 104 as one of the third external devices. In the example illustrated in FIG. 5, the external I/F 80 of the medical support device 24 is connected to the external I/F 104. The external I/F 104 controls the transmission and reception of various types of information between the processor 82 (see FIGS. 3 and 4) of the medical support device 24 and the processor 106 of the information processing device 100. For example, the information processing device 100 generates the lumen recognition model 92 (see FIGS. 4, 9, and 10), and transmits the generated lumen recognition model 92 to the medical support device 24 via the external I/Fs 80 and 104 in accordance with a request from the medical support device 24.

A machine learning processing program 120 is stored in the storage 110. The processor 106 reads out the machine learning processing program 120 from the storage 110, and executes the readout machine learning processing program 120 on the memory 108 to perform machine learning processing. Further, an example image set 122 is stored in the storage 110. Although the details will be described later, the example image set 122 is used by the processor 106.

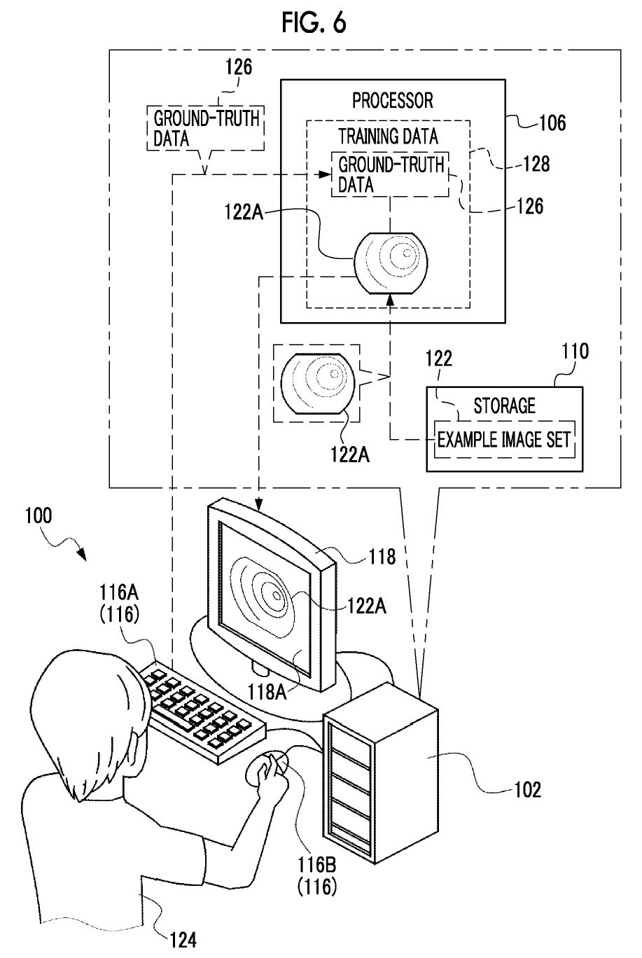

FIGS. 6 to 8 illustrate an example of the machine learning processing executed by the processor 106. As illustrated in FIG. 6 as an example, the information processing device 100 is used by an annotator 124. The annotator 124 means an operator who adds annotations for machine learning to given data (that is, an operator who performs labeling). In the example illustrated in FIG. 6, a keyboard 116A and a mouse 116B are illustrated as examples of the reception device 116. The annotator 124 gives an instruction to the computer 102 via the keyboard 116A and the mouse 116B.

The example image set 122 includes a plurality of example images 122A of different contents. The example image 122A is an image determined in advance as the medical image used for the object recognition processing (for example, processing in which the recognition unit 82A of the medical support device 24 recognizes the lumen 42 based on the frame 40 and the lumen recognition model 92). The image determined in advance as the medical image used for the object recognition processing is an image corresponding to the frame 40. Stated another way, the image corresponding to the frame 40 can also be referred to as an image that represents the frame 40. Further, the image that represents the frame 40 can also be referred to as an image showing a sample of the frame 40. Here, a first example of the image showing the sample of the frame 40 is an image obtained by actually imaging the interior portion of the large intestine with the camera. A second example of the image showing the sample of the frame 40 is a virtually created image (for example, an image generated by generative AI, such as Stable Diffusion or Midjourney).

The processor 106 acquires the example image 122A from the example image set 122 in accordance with the instruction received by the reception device 116. The processor 106 displays the example image 122A on a screen 118A of the display device 118. In a state where the example image 122A is displayed on the screen 118A, the annotator 124 indicates, to the processor 106, the lumen correspondence position that is the position in the example image 122A of the lumen shown in the example image 122A via the reception device 116. The processor 106 generates training data 128 by associating the example image 122A with ground-truth data 126 based on the lumen correspondence position indicated via the reception device 116. The association of the ground-truth data 126 with the example image 122A is implemented by adding an annotation that enables identification of the lumen correspondence position as the ground-truth data 126 to the lumen correspondence position in the example image 122A.

In this way, the processor 106 generates the plurality of pieces of training data 128 by repeatedly performing the processing of associating the ground-truth data 126 with each of the example images 122A included in the example image set 122 in accordance with the instruction given from the annotator 124.

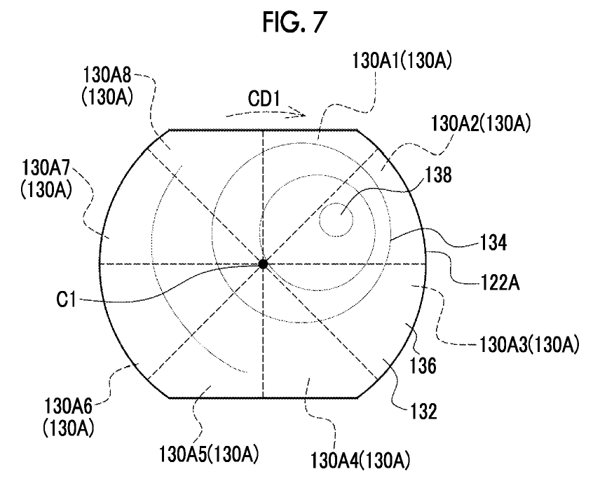

FIG. 7 is a conceptual diagram illustrating an example of a composition of the example image 122A. As illustrated in FIG. 7, a large intestine 132 is shown in the example image 122A. In the example illustrated in FIG. 7, an intestinal wall 136 in which the plurality of folds 134 are formed and a lumen 138 are shown in the example image 122A.

The example image 122A is partitioned into a plurality of partitioned regions 130A. Eight partitioned regions 130A1 to 130A8 are included in the plurality of partitioned regions 130A. The partitioned regions 130A1 to 130A8 are regions that radially exist from a center C1 of the example image 122A toward an outer edge of the example image 122A, and are disposed along a circumferential direction CD1 (in other words, about the center C1) of the example image 122A.

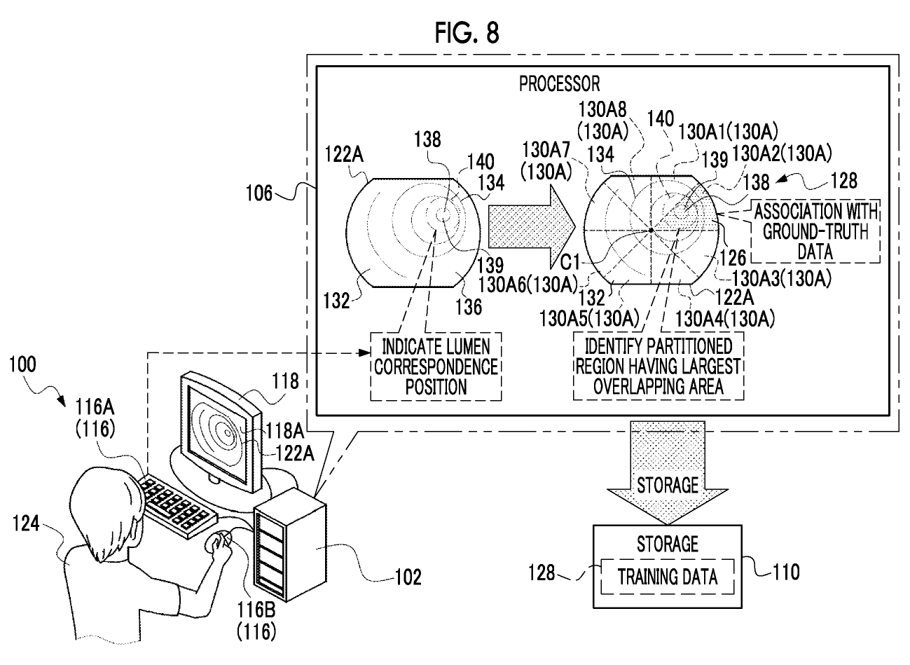

FIG. 8 is a conceptual diagram illustrating an example of a method of generating the training data 128 by associating the ground-truth data 126 with the example image 122A by the processor 106.

As illustrated in FIG. 8, in a state where the example image 122A is displayed on the screen 118A, the annotator 124 indicates, to the processor 106, the lumen correspondence position 139 that is the position in the example image 122A of the lumen 138 shown in the example image 122A via the reception device 116. The processor 106 superimposes and displays a circular frame 140 on the example image 122A in accordance with the instruction received by the reception device 116, and disposes the frame 140 at a position surrounding the lumen 138 shown in the example image 122A. The frame 140 is a mark that defines the lumen correspondence position 139 in the example image 122A. That is, a position of a region surrounded by the frame 140 in the example image 122A is the lumen correspondence position 139. The size and the position of the frame 140 are freely changed on the screen 118A in accordance with the instruction received by the reception device 116. Here, the shape of the frame 140 is a circular shape, but the shape may be a shape other than the circular shape. The size of the frame 140 can be changed in accordance with the instruction received by the reception device 116.

The annotator 124 instructs, via the reception device 116, the processor 106 to confirm the lumen correspondence position 139 that is an instruction to confirm the lumen correspondence position 139 in a state where the frame 140 is disposed at a position surrounding the lumen 138. As a result, the processor 106 confirms the lumen correspondence position 139.

The processor 106 identifies the partitioned region 130A having the largest area overlapping the frame 140 that defines the lumen correspondence position 139 from among the plurality of partitioned regions 130A. Then, the processor 106 generates the training data 128 by associating the ground-truth data 126 with the identified partitioned region 130A (in the example illustrated in FIG. 8, the partitioned region 130A2) as the annotation that enables identification of the partitioned region 130A in which the lumen 138 is shown. The processor 106 stores the generated training data 128 in the storage 110.

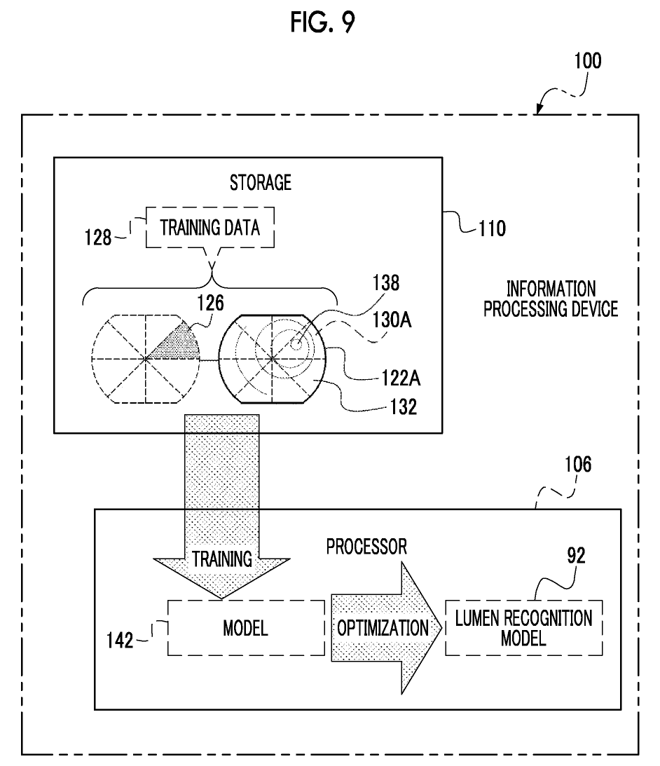

FIG. 9 is a conceptual diagram illustrating an example of an aspect in which the lumen recognition model 92 is generated by performing the machine learning using the training data 128 by the processor 106.

As illustrated in FIG. 9, in the information processing device 100, the processor 106 acquires the training data 128 from the storage 110. The processor 106 executes the machine learning using the training data 128.

In this case, for example, the processor 106 generates the lumen recognition model 92 by optimizing a model 142 using the training data 128 by using an error backpropagation method. That is, the example image 122A is input to the model 142, and a plurality of optimization variables in the model 142 are adjusted to minimize an error by comparing an output result from the model 142 with the ground-truth data 126. Examples of the plurality of optimization variables include a weight indicating strength of the connection between neurons (in other words, a connection weight), and a bias that is a value for controlling activation of a neuron (in other words, a value used to adjust an output of the neuron) (in other words, an offset value).

The model 142 is optimized by repeatedly performing the learning processing using the plurality of pieces of training data 128 by the processor 106. The lumen recognition model 92 generated by optimizing the model 142 in this way is transmitted from the information processing device 100 to the medical support device 24 via the external I/Fs 80 and 104 (see FIG. 5), and is received by the medical support device 24. In the medical support device 24, the lumen recognition model 92 is stored in the storage 86 by the processor 82 (see FIG. 4). The lumen recognition model 92 stored in the storage 86 is used by the recognition unit 82A (see FIG. 4). In the present embodiment, the lumen recognition model 92 is an example of a "trained model" according to the present disclosure.

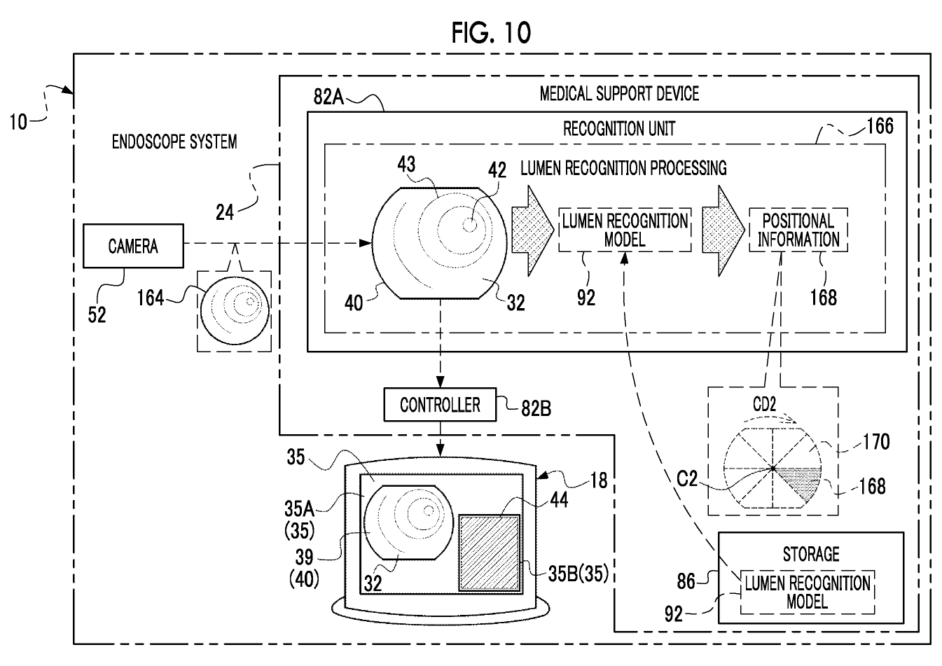

FIG. 10 illustrates an example of processing contents in the recognition unit 82A. As illustrated in FIG. 10, an image 164 obtained by imaging the intestinal wall 32 in the large intestine 28 including the lumen 42 with the camera 52 is acquired by the recognition unit 82A. The recognition unit 82A generates the frame 40 by executing various types of processing on the image 164. In the example illustrated in FIG. 10, the intestinal wall 32 having the folds 43 and the lumen 42 are shown in the frame 40.

The controller 82B acquires the frame 40 from the recognition unit 82A, and displays the acquired frame 40 in the first display region 35A. The plurality of frames 40 arranged in the time series are sequentially displayed in the first display region 35A at a predetermined frame rate, so that the plurality of frames 40 arranged in the time series are displayed as the endoscopic video image 39 in the first display region 35A.

The recognition unit 82A executes lumen recognition processing 166 on the frame 40. The lumen recognition processing 166 is processing of recognizing the lumen 42 shown in the frame 40 using the lumen recognition model 92 stored in the storage 86 (in other words, processing of identifying the existence position of the lumen 42, which is shown in the frame 40, in the frame 40 using the lumen recognition model 92).

The recognition unit 82A inputs the frame 40 to the lumen recognition model 92 to cause the lumen recognition model 92 to generate positional information 168. The recognition unit 82A holds the positional information 168 generated in the lumen recognition model 92 along the time series. For example, the positional information 168 is held by a FIFO method. The positional information 168 is information that enables identification of the lumen position. The lumen position refers to the position in the frame 40 of the lumen 42 included in the large intestine 28 shown in the frame 40. The positional information 168 is information that enables identification of a position of any one of the eight partitioned regions 170 as a position in which the lumen 42 is shown. The eight partitioned regions 170 are obtained by partitioning the frame 40 into eight parts in the same manner as the eight partitioned regions 130A are obtained. The eight partitioned regions 170 are regions that are present radially from a center C2 of the frame 40 toward an outer edge of the frame 40, and are arranged at intervals of 45 degrees along a circumferential direction CD2 (in other words, about the center C2) of the frame 40. In the present embodiment, the positional information 168 is an example of "positional information" according to the present disclosure.

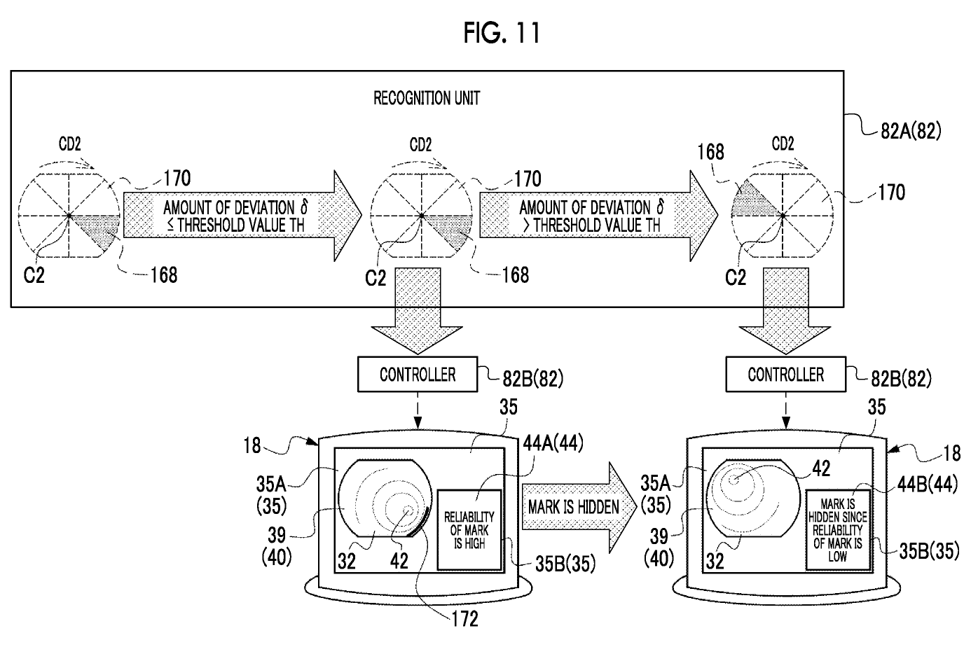

As illustrated in FIG. 11 as an example, the processor 82 generates and outputs an arc-shaped mark 172 that is visible information that enables visual identification of the position of the lumen 42 in the frame 40 on the screen 35, based on the positional information 168. The output of the mark 172 is achieved by displaying the mark 172 on the screen 35 of the display device 18.

The mark 172 is formed at a position corresponding to the position of one partitioned region 170 selected from among the plurality of partitioned regions 170 based on the positional information 168. Further, a shape of the mark 172 is a shape along an arc on a circumference having a peripheral angle of 45 degrees with respect to the center C2. The mark 172 in the present embodiment is an example of "visible information" according to the present disclosure.

In the example illustrated in FIG. 11, the recognition unit 82A calculates an amount of deviation δ between the positions of the lumen 42 identified from the positional information 168 adjacent in time each time the positional information 168 is generated and held. Then, the recognition unit 82A determines whether or not to display or not to display the mark 172 on the screen 35 based on the calculated amount of deviation δ.

Here, the amount of deviation δ is an amount of deviation (for example, an angle) about the center C2 of the frame 40 between the position of the lumen 42 identified from the reference positional information that is the reference positional information 168 and the position of the lumen 42 identified from the subsequent positional information that is the positional information 168 generated after the reference positional information. The reference positional information and the subsequent positional information are obtained by executing the lumen recognition processing 166 on each of two frames 40 adjacent in time. That is, the reference positional information is obtained by executing the lumen recognition processing 166 on the frame 40 generated earlier among the two frames 40 adjacent in time, and the subsequent positional information is obtained by executing the lumen recognition processing 166 on the frame 40 generated later among the two frames 40 adjacent in time.

The recognition unit 82A determines to display the mark 172 on the screen 35 in a case where the amount of deviation δ is equal to or less than a threshold value TH (for example, 90 degrees). The recognition unit 82A determines not to display the mark 172 on the screen 35 in a case where the amount of deviation δ exceeds the threshold value TH. The case where the amount of deviation δ exceeds the threshold value TH refers to a case where a first partitioned region that is a partitioned region to which the position of the lumen 42 identified from the reference positional information belongs among the eight partitioned regions 170 and a second partitioned region that is a partitioned region to which the position of the lumen 42 identified from the subsequent positional information belongs among the eight partitioned regions 170 deviate from each other by an amount exceeding the threshold value TH. The amount of deviation δ in the present embodiment is an example of an "amount of deviation" according to the present disclosure.

Examples of the threshold value TH include a value derived in advance by a test using an actual machine and/or computer simulation or the like as a maximum value of a shake amount of the mark 172 superimposed and displayed on the frame 40 (that is, the endoscopic video image 39) displayed on the screen 35 in time series, which does not cause the doctor 12 to be confused. The threshold value TH in the present embodiment is an example of a "threshold value" according to the present disclosure.

The controller 82B controls the display of the mark 172 on the screen 35 in accordance with the determination result of the recognition unit 82A. In the example illustrated in FIG. 11, in a case where the amount of deviation δ is equal to or less than the threshold value TH, the controller 82B superimposes and displays the mark 172 on the frame 40 displayed in the first display region 35A. The position at which the mark 172 is displayed on the frame 40 is the position of the lumen 42 identified from the subsequent positional information.

In addition, in the example illustrated in FIG. 11, the state transitions from a state where the amount of deviation δ is equal to or less than the threshold value TH to a state where the amount of deviation δ exceeds the threshold value TH. This is because the recognition unit 82A calculates the amount of deviation δ by using the positional information 168 used to generate the mark 172 superimposed and displayed on the frame 40 as the reference positional information and using the positional information 168 generated next to the reference positional information as the subsequent positional information, and determines that the calculated amount of deviation δ exceeds the threshold value TH. In this case, the controller 82B removes the mark 172 (that is, hides the mark 172) from the frame 40.

In the example illustrated in FIG. 11, the form example has been described in which the mark 172 is superimposed and displayed on the frame 40, but this is merely an example, and the mark 172 may be displayed outside the frame 40. In addition, the shape and/or the size of the mark 172 can also be changed. For example, the pattern and/or the color or the like may be applied to a part or the entirety of the partitioned region 170 in which the lumen 42 is present, or a part or the entirety of the edge of the partitioned region 170 in which the lumen 42 is present may be bordered.

In addition, in the example illustrated in FIG. 11, the form example has been described in which the mark 172 is hidden in a case where the amount of deviation δ exceeds the threshold value TH, but this is merely an example. For example, the mark 172 may be displayed in the first display region 35A in a case where the amount of deviation δ exceeds the threshold value TH. However, the mark 172 displayed in the first display region 35A in a case where the amount of deviation δ exceeds the threshold value TH is displayed in an aspect in which the visibility is lowered compared to the visibility of the mark 172 displayed in the first display region 35A in a case where the amount of deviation δ is equal to or less than the threshold value TH.

Here, as the aspect in which the visibility is lowered, there are the following first to sixth aspects. The first aspect is an aspect in which the mark 172 is displayed in a line type that is less conspicuous than the mark 172 displayed in the first display region 35A in a case where the amount of deviation δ exceeds the threshold value TH. The second aspect is an aspect in which the mark 172 is displayed in a thickness that is less conspicuous than the mark 172 displayed in the first display region 35A in a case where the amount of deviation δ exceeds the threshold value TH. The third aspect is an aspect in which the mark 172 is displayed in a color that is less conspicuous than the mark 172 displayed in the first display region 35A in a case where the amount of deviation δ exceeds the threshold value TH. The fourth aspect is an aspect in which the mark 172 is displayed in a brightness that is less conspicuous than the mark 172 displayed in the first display region 35A in a case where the amount of deviation δ exceeds the threshold value TH. The fifth aspect is an aspect in which the mark 172 is displayed in a density or a transparency that is less conspicuous than the mark 172 displayed in the first display region 35A in a case where the amount of deviation δ exceeds the threshold value TH. The sixth aspect is an aspect in which the mark 172 blinks.

In a case where the amount of deviation δ exceeds the threshold value TH, the controller 82B may not display the mark 172 on the display device 18.

As described above, in a case where the amount of deviation δ exceeds the threshold value TH, the processor 82 may suppress the output (here, as an example, the display) of the mark 172.

In addition, the processor 82 outputs reliability identification information that enables visual identification of reliability of the mark 172 (in other words, reliability of the positional information 168 generated by the recognition unit 82A) based on the amount of deviation δ. The reliability identification information is displayed in the second display region 35B by the controller 82B. The reliability identification information may be stored in a storage medium (for example, the storage 86 and/or a memory card) in association with the corresponding frame 40, may be output as audio, or may be printed by a printer.

In the example illustrated in FIG. 11, high-reliability information 44A and low-reliability information 44B are illustrated as examples of the reliability identification information. Both the high-reliability information 44A and the low-reliability information 44B are information included in the auxiliary information 44. The high-reliability information 44A is displayed in the second display region 35B as the information included in the auxiliary information 44 in a case where the amount of deviation δ is equal to or less than the threshold value TH, and the low-reliability information 44B is displayed in the second display region 35B as the information included in the auxiliary information 44 in a case where the amount of deviation δ exceeds the threshold value TH.

The high-reliability information 44A is information that enables visual identification of the fact that the reliability of the mark 172 is equal to or higher than a certain level (for example, the amount of deviation δ is equal to or less than the threshold value TH). In the example illustrated in FIG. 11, the high-reliability information 44A is displayed in the second display region 35B as text indicating that the reliability of the mark 172 is equal to or higher than a certain level.

The low-reliability information 44B is information that enables visual identification of the fact that the reliability of the mark 172 is lower than a certain level (for example, the amount of deviation δ exceeds the threshold value TH). In the example illustrated in FIG. 11, the low-reliability information 44B is displayed in the second display region 35B as text indicating that the mark 172 is hidden because the reliability of the mark 172 is lower than a certain level.

Here, a message assuming that the mark 172 is hidden is illustrated as the low-reliability information 44B, but this is merely an example. For example, even in a case where the mark 172 is displayed in the first display region 35A in any one of the first to sixth aspects described above in a case where the amount of deviation δ exceeds the threshold value TH, the low-reliability information 44B may be displayed in the second display region 35B as a message.

In this case, the visibility of the mark 172 displayed in the first display region 35A in a case where the amount of deviation δ exceeds the threshold value TH is lower than the visibility of the mark 172 displayed in the first display region 35A in a case where the amount of deviation δ is equal to or less than the threshold value TH. Then, as the low-reliability information 44B, a message indicating that the reliability of the mark 172 displayed in the first display region 35A is lower than a certain level is displayed in the second display region 35B. In addition, since the reliability of the mark 172 displayed in the first display region 35A is lower than a certain level, the low-reliability information 44B may be a message indicating that the visibility of the mark 172 is lowered.

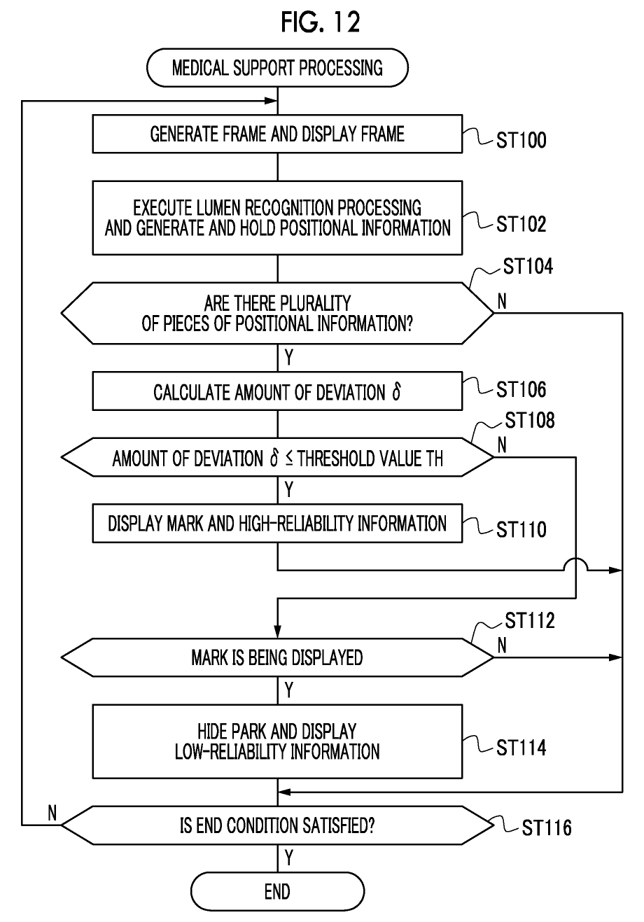

Next, an example of a flow of the medical support processing performed by the endoscope system 10 will be described with reference to FIG. 12. A flow of the medical support processing illustrated in FIG. 12 is an example of a "medical support method" according to the present disclosure.

In the medical support processing illustrated in FIG. 12, first, in step ST100, the recognition unit 82A acquires the image 164 from the camera 52, and performs various types of processing on the acquired image 164 to generate the frame 40 (see FIG. 10). Then, the controller 82B displays the latest frame 40 generated by the recognition unit 82A in the first display region 35A. After the processing in step ST100 is executed, the medical support processing advances to step ST102.

In step ST102, the recognition unit 82A executes the lumen recognition processing 166 using the lumen recognition model 92 to cause the lumen recognition model 92 to generate the positional information 168 (see FIG. 10). Then, the recognition unit 82A holds the positional information 168 along the time series in a FIFO method. After the processing in step ST102 is executed, the medical support processing advances to step ST104.

In step ST104, the recognition unit 82A determines whether or not a plurality of pieces of positional information 168 (here, as an example, two or more pieces of positional information 168) are held along the time series. In step ST104, in a case where the plurality of pieces of positional information 168 are not held along the time series (for example, in a case where only one piece of positional information 168 is held), a negative determination is made, and the medical support processing advances to step ST116. In step ST104, in a case where the plurality of pieces of positional information 168 are held along the time series, an affirmative determination is made, and the medical support processing advances to step ST106.

In step ST106, the recognition unit 82A calculates the amount of deviation δ based on the plurality of pieces of positional information 168 held along the time series (see FIG. 11). Here, for example, the amount of deviation about the center C2 of the frame 40 between the positions of the lumen 42 identified from the latest two pieces of positional information 168 is calculated as the amount of deviation δ. After the processing in step ST106 is executed, the medical support processing advances to step ST108.

In step ST108, the recognition unit 82A determines whether or not the amount of deviation δ calculated in step ST106 is equal to or less than the threshold value TH. In step ST108, in a case where the amount of deviation δ is not equal to or less than the threshold value TH, a negative determination is made, and the medical support processing advances to step ST112. In step ST108, in a case where the amount of deviation δ is equal to or less than the threshold value TH, an affirmative determination is made, and the medical support processing advances to step ST110.

In step ST110, the controller 82B superimposes and displays the mark 172 that enables visual identification of the position of the lumen 42 in the frame 40 in the first display region 35A on the frame 40 displayed in the first display region 35A (see FIG. 11). The position at which the mark 172 is superimposed and displayed on the frame 40 is a position corresponding to the position of the lumen 42 identified from the latest positional information 168. In addition, the controller 82B displays the high-reliability information 44A in the second display region 35B (see FIG. 11). After the processing in step ST110 is executed, the medical support processing advances to step ST116.

In step ST112, the controller 82B determines whether or not the mark 172 is being displayed in the first display region 35A. In step ST112, in a case where the mark 172 is not being displayed in the first display region 35A, a negative determination is made, and the medical support processing advances to step ST116. In step ST112, in a case where the mark 172 is being displayed in the first display region 35A, an affirmative determination is made, and the medical support processing advances to step ST114.

In step ST114, the controller 82B hides the mark 172. That is, the controller 82B removes the mark 172 displayed in the first display region 35A (see FIG. 11). In addition, the controller 82B displays the low-reliability information 44B in the second display region 35B (see FIG. 11). After the processing in step ST114 is executed, the medical support processing advances to step ST116.

In step ST116, the controller 82B determines whether or not a medical support processing end condition is satisfied. Examples of the medical support processing end condition include a condition in which the instruction to end the medical support processing is given to the endoscope system 10 (for example, a condition in which the reception device 64 receives the instruction to end the medical support processing).

In a case where the medical support processing end condition is not satisfied in step ST116, a negative determination is made, and the medical support processing advances to step ST100. In a case where the medical support processing end condition is satisfied in step ST116, an affirmative determination is made, and the medical support processing ends.

As described above, in the endoscope system 10, the processor 82 generates the positional information 168 that enables identification of the position in the frame 40 of the lumen 42 shown in the frame 40 based on the frame 40 obtained by imaging the interior portion of the large intestine 28 with the endoscope 16. In addition, the processor 82 generates and outputs the mark 172 that enables visual identification of the position of the lumen 42 in the frame 40 on the screen 35 based on the positional information 168. Here, in a case where the position of the lumen 42 identified from the positional information 168 changes significantly each time the positional information 168 is generated, the display position of the mark 172 also changes significantly, so that there is a concern that the doctor 12 may be confused.

Therefore, in the endoscope system 10, the processor 82 suppresses the output of the mark 172 that is the visible information based on the amount of deviation δ about the center C2 of the frame 40 between the positions of the lumen 42 identified from the two pieces of positional information 168 (that is, the reference positional information and the subsequent positional information) adjacent in time. The output of the mark 172 is achieved by displaying the mark 172 on the screen 35. The suppression of the output of the mark 172 is achieved by, for example, hiding the mark 172, making the mark 172 thin, reducing the size of the mark 172, or the like (that is, reducing a visual recognition level that is a level at which the doctor 12 can visually recognize). In this way, it is possible to prevent the doctor 12 who observes the mark 172 from being confused due to a large change in the position of the lumen 42 visually identified from the mark 172 on the screen 35. As a result, it is possible for the doctor 12 to safely and efficiently advance the endoscopy and various treatments.

In the endoscope system 10, the frame 40 is input to the lumen recognition model 92 to generate the positional information 168 by the lumen recognition model 92. As described above, by executing the lumen recognition processing 166 using the lumen recognition model 92, the position of the lumen 42 is estimated with higher accuracy than in non-AI-based object recognition processing such as the template matching in the related art. On the other hand, since the lumen recognition processing 166 is the AI-based object recognition processing, there is a concern that a sudden estimation jump (for example, a phenomenon in which the display position of the mark 172 changes significantly instantaneously) may occur, but the output of the mark 172 is suppressed in the above-described manner even in a case where the positional information 168 is generated by the AI-based method using the lumen recognition model 92 (that is, in a case where the position of the lumen 42 is recognized). Therefore, even in a case where the position of the lumen 42 identified from the positional information 168 generated by the lumen recognition model 92 changes significantly, it is possible to prevent the doctor 12 who observes the mark 172 from being confused.

In addition, in the endoscope system 10, the output of the mark 172 is suppressed in a case where the amount of deviation δ exceeds the threshold value TH. Therefore, even in a case where the amount of deviation δ exceeds the threshold value TH (here, as an example, 90 degrees), the output of the mark 172 is suppressed in the above-described manner, so that it is possible to prevent the doctor 12 who observes the mark 172 from being confused.

In addition, in the endoscope system 10, the output of the mark 172 is suppressed in a case where a first partitioned region that is the partitioned region 170 to which the position of the lumen 42 identified from the reference positional information belongs among the eight partitioned regions 170 radially partitioned from the frame 40 and a second partitioned region that is the partitioned region 170 to which the position of the lumen 42 identified from the subsequent positional information belongs among the eight partitioned regions 170 deviate from each other by an amount exceeding the threshold value TH (here, as an example, 90 degrees). In this way, it is possible to reduce the processing load required for tracking the position of the lumen 42. Since the mark 172 is output or the output is suppressed in units of the partitioned regions 170, the doctor 12 can easily visually ascertain whether or not the position of the lumen 42 visually identified from the mark 172 on the screen 35 deviates from the center C2 of the frame 40 by the threshold value TH (here, as an example, 90 degrees).

In addition, in the endoscope system 10, the processor 82 outputs the reliability identification information that enables visual identification of the reliability of the mark 172 based on the amount of deviation δ. The reliability identification information is displayed in the second display region 35B as a part of the auxiliary information 44. As a result, the doctor 12 who observes the mark 172 on the screen 35 can check the reliability of the observed mark 172 through the reliability identification information. In a case where the amount of deviation δ exceeds the threshold value TH (here, as an example, 90 degrees), the low-reliability information 44B is displayed in the second display region 35B as information that enables visual identification of the fact that the reliability of the mark 172 is lower than a certain level. As a result, it is possible for the doctor 12 who observes the mark 172 on the screen 35 to easily visually ascertain that the reliability of the observed mark 172 is lower than a certain level.

In the above embodiment, the form example has been described in which the position of the lumen 42 in the frame 40 is identified by the AI method using the lumen recognition model 92, and the identified result is generated as the positional information 168, but this is merely an example. For example, the position of the lumen 42 in the frame 40 may be identified by a non-AI method (for example, template matching or pattern matching), and the identification result may be generated as the positional information 168.

In the above embodiment, the eight partitioned regions 170 have been described as an example, but this is merely an example, and there may be nine or more radially partitioned regions or less than eight radially partitioned regions.

In the above embodiment, the form example has been described in which the output of the mark 172 is suppressed (for example, the form example in which the mark 172 is hidden) in a case where the amount of deviation δ exceeds the threshold value TH, but the present disclosure is not limited to this. For example, the output of the mark 172 may be suppressed from when the amount of deviation δ exceeds the threshold value TH until the predetermined condition is satisfied. In this way, it is possible to prevent the doctor 12 who observes the mark 172 on the screen 35 from visually recognizing the mark 172 from when the amount of deviation δ exceeds the threshold value TH until the predetermined condition is satisfied. As a result, it is possible to prevent the doctor 12 from being confused due to a large change in the position of the lumen 42 visually identified from the mark 172 on the screen 35.

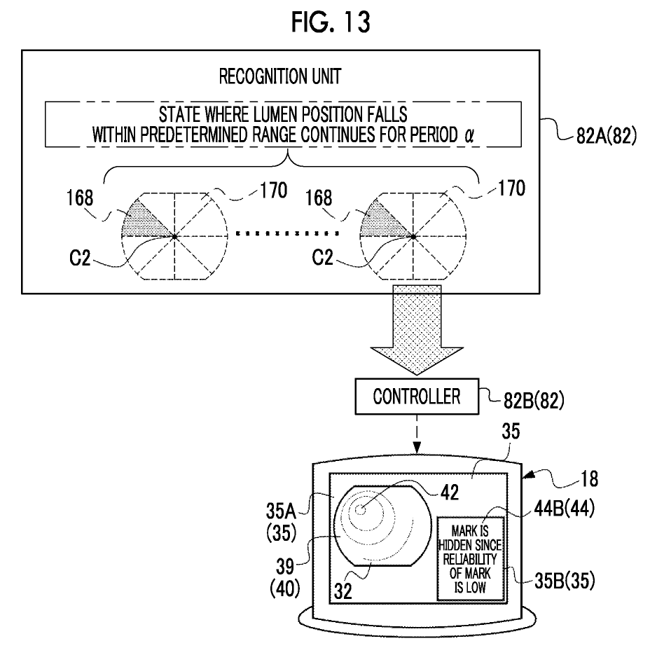

Examples of the predetermined condition include a condition in which the position of the lumen 42 identified from the positional information 168 generated based on each of the plurality of frames 40 adjacent in time is within a predetermined range (for example, within 90 degrees) about the center C2 for a designated period α, as illustrated in FIG. 13. In the example illustrated in FIG. 13, the mark 172 is hidden while the position of the lumen 42 identified from the positional information 168 generated based on each of the plurality of frames 40 adjacent in time continuously falls within the predetermined range about the center C2 for the period α. In addition, the low-reliability information 44B is displayed in the second display region 35B. In a case where the period α has elapsed, the mark 172 is superimposed and displayed on the frame 40 or the mark 172 is hidden based on the amount of deviation δ in the same manner as in the above embodiment.

By hiding the mark 172 under the condition illustrated in FIG. 13, it is possible to prevent the doctor 12 who observes the mark 172 on the screen 35 from visually recognizing the mark 172 until the position of the lumen 42 visually identified from the mark 172 on the screen 35 is stabilized. As a result, it is possible to prevent the doctor 12 from being confused due to a large change in the position of the lumen 42 visually identified from the mark 172 on the screen 35.

As a first example of the period α, a period is adopted in which the visual continuity of the mark 172 is maintained in a case where an output state (here, as an example, a display state) in which the output (here, as an example, the display) of the mark 172 is performed transitions to an output suppression state (here, as an example, a display suppression state) in which the output (here, as an example, the display) of the mark 172 is suppressed, and then is returned to the output state. The period in which the visual continuity of the mark 172 is maintained refers to, for example, a period derived in advance by a test using an actual machine and/or computer simulation or the like as a period in which the display of the mark 172 is not visually recognized as being interrupted from the display of the mark 172 corresponding to the frame 40 before the mark 172 is not displayed until the output state is returned (that is, until the display of the mark 172 is returned). By using the period α determined in this way, it is possible to prevent the doctor 12 who observes the mark 172 on the screen 35 from visually recognizing the mark 172 from being interrupted.

A second example of the period α includes a period determined based on the amount of deviation δ (for example, the amount of deviation δ calculated based on the positional information 168 corresponding to the frame 40 before the period α and the positional information 168 corresponding to the first frame 40 after the period α). It is preferable to use the period determined in this way as the period α because, in a case where the amount of deviation δ is small, the time required for the lumen position to be stabilized is also short, and in a case where the amount of deviation δ is large, the time required for the lumen position to be stabilized is likely to be long. Therefore, the period α corresponding to the amount of deviation δ may be determined in accordance with a rule-based system in which the correspondence relationship between the amount of deviation δ and the period α is determined in advance based on such a relationship. As a result, a period without excess or deficiency can be determined as the period α as compared to a case where the period α is fixed regardless of the amount of deviation δ.

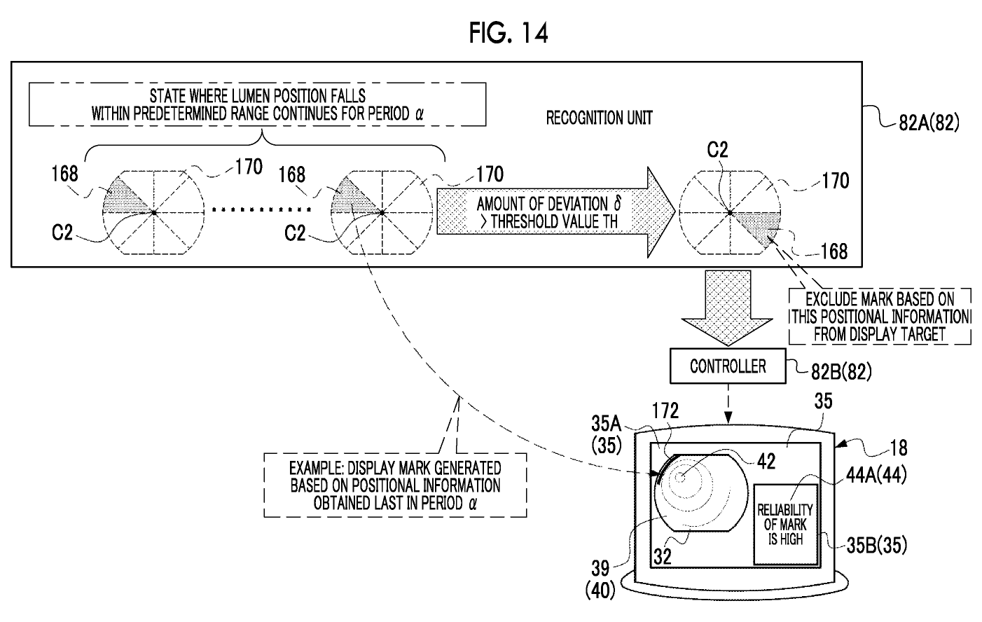

As illustrated in FIG. 14 as an example, in a case where the position of the lumen 42 identified from the positional information 168 generated based on at least one frame 40 (for example, the first frame 40) after the period α has elapsed deviates from the position of the lumen 42 identified from the positional information 168 generated in a case where the predetermined condition is satisfied (for example, the positional information 168 generated based on the last frame 40 among the plurality of pieces of positional information 168 generated within the period α) by an amount exceeding the threshold value TH about the center C2, the mark 172 generated based on the positional information 168 generated in a case where the predetermined condition is satisfied may be superimposed and displayed on the frame 40. For example, in a case where the position of the lumen 42 identified from the positional information 168 generated based on at least one frame 40 (for example, the first frame 40) after the period α has elapsed deviates from the position of the lumen 42 identified from the positional information 168 generated last in the period α by exceeding the threshold value TH about the center C2, the mark 172 based on the positional information 168 generated based on the frame 40 after the period α may not be a display target, and the mark 172 based on the positional information 168 generated last in the period α may be superimposed and displayed on the frame 40. In this way, it is possible to suppress the occurrence of a situation in which the doctor 12 is confused by causing the doctor 12 to observe the mark 172 having unstable behavior on the screen 35.

In the example illustrated in FIG. 14, the amount of deviation δ exceeding the threshold value TH is determined to be a momentary shake, and the high-reliability information 44A is displayed in the second display region 35B, but this is merely an example. For example, since the mark 172 is displayed in the first display region 35A even in a case where the amount of deviation δ exceeds the threshold value TH, the display of such a mark 172 may be determined to have low reliability, and in this case, the low-reliability information 44B may be displayed in the second display region 35B.

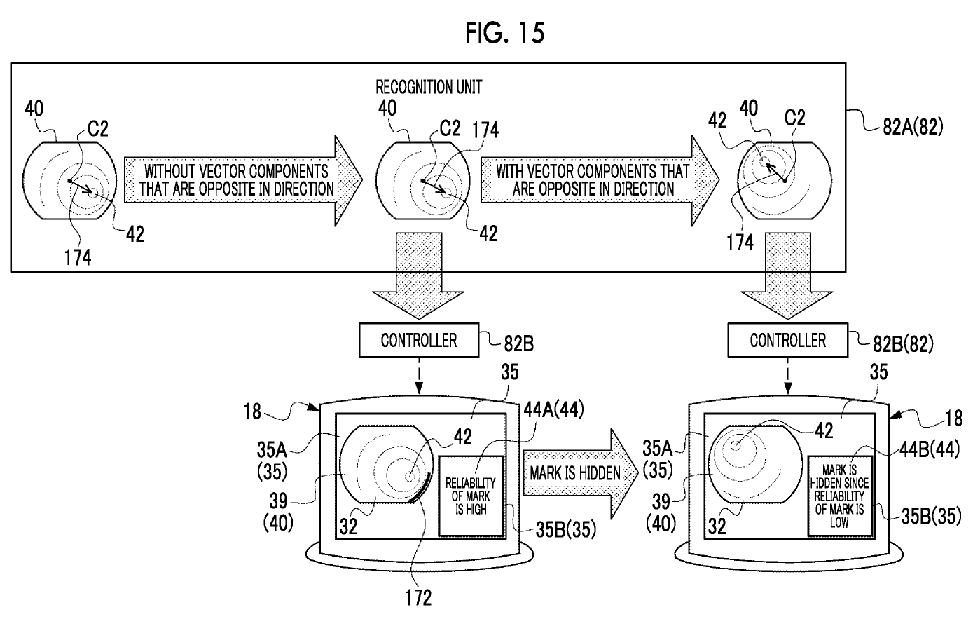

In the above embodiment, the form example has been described in which the mark 172 is hidden in a case where the first partitioned region that is the partitioned region 170 to which the position of the lumen 42 identified from the reference positional information belongs among the eight partitioned regions 170 and the second partitioned region that is the partitioned region 170 to which the position of the lumen 42 identified from the subsequent positional information belongs among the eight partitioned regions 170 deviate from each other by an amount exceeding the threshold value TH, but this is merely an example. For example, as illustrated in FIG. 15, a vector 174 may be used instead of the partitioned region 170. The vector 174 may be a vector that enables identification of a direction from the center C2 of the frame 40 toward the position of the lumen 42 identified from the positional information 168. In addition, the positional information 168 may be represented by the vector (that is, the vector 174 may be used as the positional information 168). In a case of realizing this, for example, in the training stage, ground-truth data indicated by the vector that enables identification of the direction from the center C1 (see FIG. 7) toward the lumen 138 may be used for the machine learning on the model 142 (see FIG. 9) instead of the ground-truth data 126 (see FIGS. 6, 8, and 9).

As illustrated in FIG. 15, in a case where the vector 174 is used instead of the partitioned region 170, a case where a first vector (hereinafter, simply referred to as a "first vector") that is the vector 174 that enables identification of a direction from the center C2 of the frame 40 toward the position of the lumen 42 identified from the reference positional information and a second vector (hereinafter, simply referred to as a "second vector") that is the vector 174 that enables identification of a direction from the center C2 of the frame 40 toward the position of the lumen 42 identified from the subsequent positional information do not have vector components that are opposite in direction has the same meaning as a case where "amount of deviation δ ≤ threshold value TH (= 90 degrees)", so that the mark 172 is superimposed and displayed on the frame 40 displayed in the first display region 35A in the same manner as in the example illustrated in FIG. 11. In addition, a case where the first vector and the second vector have vector components that are opposite in direction has the same meaning as a case where "amount of deviation δ > threshold value TH (= 90 degrees)", so that the mark 172 superimposed and displayed on the frame 40 is hidden in the same manner as in the example illustrated in FIG. 11. As described above, even in a case where it is determined whether the amount of deviation δ ≤ threshold value TH (= 90 degrees) or the amount of deviation δ > threshold value TH (= 90 degrees) by using the vector 174 instead of the partitioned region 170, and the display and the hiding of the mark 172 are switched in accordance with the determination result, the same effect as in the above embodiment can be obtained.

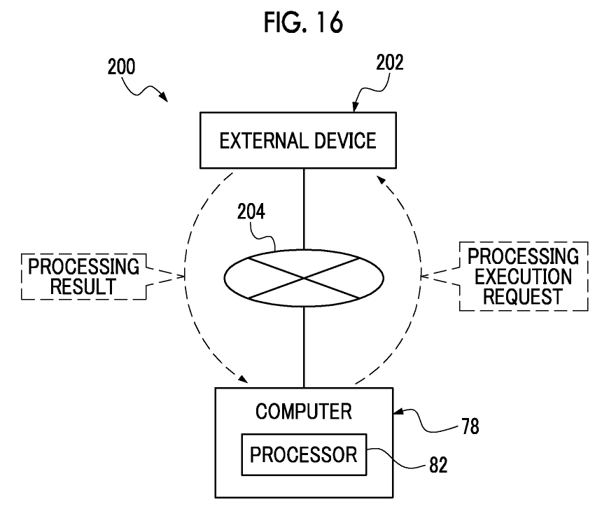

In the above embodiment, the form example has been described in which the medical support processing is executed by the computer 78, but the present disclosure is not limited to this, and at least a part of the medical support processing may be executed by a device provided outside the computer 78. Hereinafter, an example of this case will be described with reference to FIG. 16.

FIG. 16 is a conceptual diagram illustrating an example of a configuration of an endoscope system 200. The endoscope system 200 is an example of an "endoscope system" according to the present disclosure. The endoscope system 200 is different from the endoscope system 10 according to the above-described embodiment in that an external device 202 is included.

The external device 202 is connected communicably to the computer 78 via a network 204 (for example, a WAN and/or a LAN).

Examples of the external device 202 include at least one server that directly or indirectly transmits and receives data to and from the computer 78 via the network 204. The external device 202 receives a processing execution instruction given from the processor 82 of the computer 78 via the network 204. Then, the external device 202 executes processing corresponding to the received processing execution instruction, and transmits the processing result to the computer 78 via the network 204. In the computer 78, the processor 82 receives the processing result transmitted from the external device 202 via the network 204, and executes the processing using the received processing result.

Examples of the processing execution instruction include an instruction for the external device 202 to execute at least a part of the medical support processing.

A first example of the at least a part of the medical support processing (that is, processing to be executed by the external device 202) is the lumen recognition processing 166. In this case, the external device 202 executes the lumen recognition processing 166 in accordance with the processing execution instruction given from the processor 82 via the network 204, and transmits the positional information 168 to the computer 78 via the network 204. In the computer 78, the processor 82 receives the positional information 168, and executes processing using the received positional information 168.

A second example of at least a part of the medical support process (that is, the processing executed by the external device 202) includes processing by the controller 82B (for example, the processing described in the above embodiment). In this case, the external device 202 executes the processing by the controller 82B in accordance with the processing execution instruction given from the processor 82 via the network 204, and transmits the processing result (for example, the visible information to be displayed on the screen 35) to the computer 78 via the network 204. In the computer 78, the processor 82 receives the processing result and executes the same processing as the processing in the above embodiment using the received processing result.

The external device 202 may be implemented by cloud computing. The cloud computing is merely an example, and the external device 202 may be implemented by network computing, such as fog computing, edge computing, or grid computing.

In each of the above embodiment, each processing is executed by any computer. Any computer may execute these processes by a processor as hardware, a program as software, or a combination of the processor and the program. In such a case, the processor is configured to execute various types of processing in the above embodiment in cooperation with the program, and may function as each unit or each means in the present embodiment. The execution order of the processing by the processor is not limited to the above-described order and may be changed as appropriate. Any computer may be a general-purpose computer, a computer for a specific use, a workstation, or another system that can execute each processing.

The processor may be configured by one or more hardware components, and the types of hardware components are not limited. The processor may be configured by, for example, hardware such as a CPU, an MPU, a dedicated circuit for executing specific processing, such as a PLD (for example, an SPLD, a CPLD, or an FPGA), or an ASIC, a GPU, or an NPU. Further, the type of hardware component may be a combination of different types of hardware components. In a case where the plurality of types of hardware components are configured to execute one or a plurality of processes of a certain processor, the plurality of types of hardware components may be present in devices physically separated from each other or may be present in the same device. Further, in any of the embodiments, the order of each process performed by the processor is not limited to the order described above, and may be changed as appropriate. The hardware is configured by an electrical circuit (circuitry) in which circuit elements, such as semiconductor elements, are combined.

Further, the program may be software such as firmware or microcode. Additionally, the program may be, for example, a program module group, and each function thereof may be executed by the processor configured to execute the corresponding function. The program may be a program code or a plurality of code segments stored in one or a plurality of non-transitory computer-readable media (for example, a storage medium and/or other storages). The program may be distributed and stored across a plurality of non-transitory computer-readable media existing in devices physically separated from each other. The program code or the code segment may represent any combination of procedures, functions, subprograms, routines, subroutines, modules, software packages, classes, or commands, data structures, or program statements. The program code or the code segment may be connected to another code segment or a hardware circuit by transmitting and receiving information, data, an argument, a parameter, or a content of a memory.

In addition, in the above embodiment, the form example has been described in which the medical support program 90 is stored in the storage 86 in advance (that is, the form example in which the medical support program 90 is installed), but the present disclosure is not limited to this. The medical support program may be provided in a form stored in a storage medium such as a CD-ROM, a DVD-ROM, and a USB memory. Further, the medical support program 90 may be downloaded from an external device via a network.

The technology of the present disclosure extends to any program products. The program product includes all forms of products for providing the program. For example, the program product includes a program provided through a network such as the Internet, and a non-transitory computer-readable recording medium such as a CD-ROM, a DVD, or a USB memory in which the program is stored.

The above medical support processing is merely an example. Therefore, it goes without saying that unnecessary steps may be deleted, new steps may be added, or the processing order may be changed, within a range that does not deviate from the gist of the present disclosure.

The above-described contents and the above-illustrated contents are the detailed description of the parts according to the present disclosure, and are merely examples of the present disclosure. For example, the descriptions of the configurations, the functions, the operations, and the effects are the descriptions of the examples of the configurations, the functions, the operations, and the effects of the parts according to the present disclosure. Therefore, it goes without saying that unnecessary parts may be deleted, new elements may be added, or replacements may be made with respect to the above-described contents and the above-illustrated contents within a range that does not deviate from the gist of the present disclosure. In order to avoid confusion and to facilitate understanding of the parts according to the present disclosure, the description of common technical knowledge or the like, which does not particularly require the description for enabling the implementation of the present disclosure, is omitted in the above-described contents and the above-illustrated contents.

All of the documents, the patent applications, and the technical standards described in the present specification are incorporated into the present specification by reference to the same extent as in a case where each of the documents, the patent applications, and the technical standards are specifically and individually stated to be described by reference.

What is claimed is:

1. A medical support device comprising:
a processor configured to:
generate, based on an image obtained by imaging an interior portion of a luminal organ, positional information that enables identification of a lumen position that is a position in the image of a lumen shown in the image;
output visible information that enables visual identification of the lumen position in the image on a screen based on the positional information; and
suppress output of the visible information based on an amount of deviation about a center of the image between the lumen position identified from reference positional information that is the positional information and the lumen position identified from subsequent positional information that is the positional information generated after the reference positional information.

2. The medical support device according to claim 1,
wherein the positional information is generated by a trained model by inputting the image to the trained model.

3. The medical support device according to claim 1,
wherein the output of the visible information is suppressed in a case where the amount of deviation exceeds a threshold value.

4. The medical support device according to claim 3,
wherein the output of the visible information is suppressed from when the amount of deviation exceeds the threshold value until a predetermined condition is satisfied.

5. The medical support device according to claim 4,
wherein the predetermined condition includes a condition in which the lumen position identified from each piece of the positional information generated based on each of a plurality of the images adjacent in time falls within a predetermined range about the center for a designated period.

6. The medical support device according to claim 5,
wherein the period is a period during which visual continuity of the visible information is maintained in a case where an output state in which the visible information is output transitions to an output suppression state in which the output of the visible information is suppressed, and then is returned to the output state.

7. The medical support device according to claim 5,
wherein the period is determined based on the amount of deviation.

8. The medical support device according to claim 4,
wherein the processor is configured to output the visible information based on the positional information generated in a case where the predetermined condition is satisfied, in a case where the lumen position identified from the positional information generated after the predetermined condition is satisfied deviates from the lumen position identified from the positional information generated in a case where the predetermined condition is satisfied by an amount exceeding the threshold value about the center.

9. The medical support device according to claim 3,
wherein the threshold value is 90 degrees.

10. The medical support device according to claim 3,
wherein the case where the amount of deviation exceeds the threshold value refers to a case where a first vector that enables identification of a direction from the center toward the lumen position identified from the reference positional information and a second vector that enables identification of a direction from the center toward the lumen position identified from the subsequent positional information have vector components that are opposite in direction.

11. The medical support device according to claim 3,
wherein the case where the amount of deviation exceeds the threshold value refers to a case where a first partitioned region that is a partitioned region to which the lumen position identified from the reference positional information belongs among a plurality of partitioned regions obtained by partitioning the image along the center and a second partitioned region that is a partitioned region to which the lumen position identified from the subsequent positional information belongs among the plurality of partitioned regions deviate from each other by an amount exceeding 90 degrees.

12. The medical support device according to claim 11,
wherein each of the plurality of partitioned regions is a region obtained by radially partitioning the image.

13. The medical support device according to claim 12,
wherein the plurality of partitioned regions are eight radially partitioned regions.

14. The medical support device according to claim 1,
wherein the processor is configured to output reliability identification information that enables visual identification of reliability of the visible information based on the amount of deviation.

15. The medical support device according to claim 14,
wherein the processor is configured to output information that enables visual identification of a fact that the reliability is lower than a certain level as the reliability identification information in a case where the amount of deviation exceeds the threshold value.

16. The medical support device according to claim 1,
wherein the output of the visible information is achieved by displaying the visible information on the screen.

17. The medical support device according to claim 1,
wherein the image is an endoscopic image generated by imaging the interior portion of the luminal organ including the lumen with an endoscope.

18. An endoscope system comprising:
the medical support device according to claim 1; and
an endoscope,
wherein the image is generated by imaging the interior portion of the luminal organ including the lumen with the endoscope.

19. A medical support method comprising:
generating, based on an image obtained by imaging an interior portion of a luminal organ, positional information that enables identification of a lumen position that is a position in the image of a lumen shown in the image;
outputting visible information that enables visual identification of the lumen position in the image on a screen based on the positional information; and
suppressing output of the visible information based on an amount of deviation about a center of the image between the lumen position identified from reference positional information that is the positional information and the lumen position identified from subsequent positional information that is the positional information generated after the reference positional information.

20. A non-transitory computer-readable storage medium storing a program executable by a computer to execute comprising:
generating, based on an image obtained by imaging an interior portion of a luminal organ, positional information that enables identification of a lumen position that is a position in the image of a lumen shown in the image;
outputting visible information that enables visual identification of the lumen position in the image on a screen based on the positional information; and
suppressing output of the visible information based on an amount of deviation about a center of the image between the lumen position identified from reference positional information that is the positional information and the lumen position identified from subsequent positional information that is the positional information generated after the reference positional information.
